(19) United States
(12) Patent Application Publication
(10) Pub. No.: US 2014/0113964 A1
(43) Pub. Date: Apr. 24, 2014

(54) METHOD OF PREVENTING AND TREATING ALLERGIC AIRWAY DISEASES VIA INTRANASAL GABEXATE MESILATE

(71) Applicants: MEY-FANN LEE, Taichung (TW); YI-HSING CHEN, Taichung (TW); CHIA-WEI CHANG, Taichung (TW)

(72) Inventors: MEY-FANN LEE, Taichung (TW); YI-HSING CHEN, Taichung (TW); CHIA-WEI CHANG, Taichung (TW)

(73) Assignee: TAICHUNG VETERANS GENERAL HOSPITAL, TAICHUNG (TW)

(21) Appl. No.: 13/658,281

(22) Filed: Oct. 23, 2012

(51) Int. Cl. A61K 31/24; A61P 11/06; A61P 37/08

(52) U.S. Cl. 514/533

(57) ABSTRACT

The present invention discloses a method of preventing and treating allergic airway diseases via intranasal gabexate mesilate, comprising administrating a pharmaceutically effective amount of a composition comprising a gabexate mesilate via nasal spray to prevent and treat allergy induced by a serine protease type allergen.

### BACKGROUND OF THE INVENTION

1. Field of the Invention

The present invention relates to a method of preventing and treating allergy, particularly to a method of preventing and treating allergy induced by a serine protease type allergen through administering gabexate mesilate via nasal spray.

2. Description of the Related Art

An allergy is a hypersensitivity disorder of the immune system. Allergic reactions occur when a person's immune system reacts to normally harmless substances in the environment. A substance that causes a reaction is called an allergen. These reactions are acquired, predictable, and rapid. Allergy is one of four forms of hypersensitivity and is formally called type I (or immediate) hypersensitivity. Allergic reactions are distinctive because of excessive activation of certain white blood cells called mast cells and basophils by a type of antibody called Immunoglobulin E (IgE). Moreover, the symptoms of allergy may comprise sneezing, runny or clogged nose, coughing, postnasal drip, itching eyes, nose, and throat, allergic shiners, watering eyes, conjunctivitis, asthma etc.

Among various allergens, the airborne allergens with proteolytic activity have been demonstrated to have a direct effect on human airway epithelial cells via activation of protease-activated receptors (PARs). For instance, the allergens may be from mite, animals, mold etc. Wherein, Lee et al. demonstrated that American cockroach allergens (CraA) activate the PARS of A549 cells, a lung epithelial cell, and thereby phosphorylate ERK1/2 and JNK kinase in the MAP kinase signaling pathway, generate IL-8 mRNA, release proteins and consequently result in inflammation of airway.

Clinically, two kinds of drugs, β₂-adrenergic agonists and corticosteroids, are often used for treating asthma. Using the combination of the drugs can effectively alleviate the inflammation and contraction of bronchus caused by asthma. However, these two kinds of drugs may not effectively treat some of the asthma patients due to individual differences. In addition, although high dose of corticosteroids may control the inflammation caused by allergy, the high dose of corticosteroids would cause many side effects, such as cataract and osteoporosis, as well.

Gabexate mesilateis, a serine protease inhibitor, is generally used in the therapeutic treatment of pancreatitis, disseminated intravascular coagulation, and as a regional anticoagulant for hemodialysis. It was demonstrated that intra-peritoneal injection of gabexate mesilateis attenuates allergen-induced airway inflammation and eosinophilia in a murine asthma model.

### SUMMARY OF THE INVENTION

In view of the shortcomings of the prior art, it is a primary objective of the present invention to provide a method of preventing and treating allergy, specifically a method of preventing and treating allergy induced by a serine protease type allergen though administering gabexate mesilate via nasal spray.

To achieve the aforementioned objective, the present invention provides a method of preventing and treating allergic airway diseases via intranasal gabexate mesilate, comprising administrating a pharmaceutically effective amount of a composition comprising a gabexate mesilate via nasal spray to prevent and treat allergy induced by a serine protease type allergen.

Preferably, the serine protease type allergen may comprise a serine protease activity.

Preferably, the serine protease inhibitor may prevent or inhibit the serine protease type allergen from activating of a protease-activated receptor (PAR).

Preferably, the PAR may comprise PAR-2 and PAR-3.

Preferably, the serine protease type allergen may induce a dissociation of epithelium cells from an extracellular matrix via the serine protease activity.

Preferably, a source of the serine protease type allergen may comprise mite, mold, cat and cockroach.

Preferably, the serine protease type allergen being from mite may comprise Der p 3, Der p 6 and Der p 9.

Preferably, the serine protease type allergen being from cockroach may comprise American cockroach allergens (CraA).

Preferably, the composition may further comprise distilled water, phosphate buffer solution (PBS) and the like.

Preferably, the pharmaceutically effective amount of the composition may be between 1-600 μg/mL.

Preferably, the allergic airway diseases may comprise sneezing, runny or clogged nose, postnasal drip, itching nose and asthma.

The method of preventing and treating allergy have one or more of the following advantages:

(1) The method of preventing and treating allergy in accordance with the present invention can topically apply the composition comprising gabexate mesilate in nasal mucosa to avoid the dissociation of the cellular barrier, epithelial cells, thereby protect cellular barrier from the serine type allergens.

(2) The method of preventing and treating allergy in accordance with the present invention can topically apply gabexate mesilate via nasal spray to prevent or treat the allergy induced by serine type allergens instead of suppressing the immuno-inflammatory cascade.

(3) The method of preventing and treating allergy in accordance with the present invention can reduce the side effects of the existing anti-inflammatory drugs, such as β₂-adrenergic agonists and corticosteroids.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic view of inhibition of protease activity of CraA in accordance with an embodiment of the present invention.

FIG. 2 is a schematic view of Beas2B cells treated with various doses of CraA in accordance with an embodiment of the present invention.

FIG. 3 is a western blot of PAR-2 and PAR-3 protein in Beas2B cells treated with CraA in accordance with an embodiment of the present invention.

FIG. 4 is a quantification result of chemokines detected by RT-PCR in Beas2B cells treated with CraA in various time points in accordance with an embodiment of the present invention.

FIG. 5 is a schematic view of cell viability of Beas2B cells treated with gabexate mesilate in accordance with an embodiment of the present invention.

FIG. 6 is a schematic view of protection effects of gabexate mesilate on Beas2B cells treated with CraA in accordance with an embodiment of the present invention.

FIG. 7 is a schematic view of chemokines detected by RT-PCR in Beas2B cells treated with CraA and gabexate mesilate in accordance with an embodiment of the present invention.

FIG. 8 is a quantification result of IL-8 and MCP-1 proteins in Beas2B cells treated with CraA and gabexate mesilate in accordance with an embodiment of the present invention.

### DESCRIPTION OF THE PREFERRED EMBODIMENTS

The technical content of the present invention will become apparent by the detailed description of the following embodiments and the illustration of related drawings as follows. The embodiments may be in different forms and should not be construed as limited to the embodiments set forth herein.

Establishment of Beas2B Cell Model

Human bronchus epithelial cells, Beas2B cells, were used in the present invention to mimic the in vivo environment of the bronchus. In an embodiment, American cockroach allergens (CraA) were used as serine protease type allergens in the present invention to induce immuno-inflammatory cascade and dissociation. The serine protease type allergens may further comprise Der p 3, Der p 6 and Der p 9 from mite, and any other serine protease type allergens from mold, cat etc.

In order to demonstrate that CraA used in the present invention possess serine protease activity, azocasein assay was applied to measure the protease activity of CraA. In brief, azocasein was used as a substrate to measure the protease activity of CraA. Twenty μL of CraA was added into 80 μL azocasein solution (0.5% (w/v), lysed in 100 mM phosphate buffer) and incubated in 37° C. water bath for 30 mins. Afterwards, 20 μL of 10% trichloroacetic acid was added to stop the reaction. Further, the mixture was centrifuged with 6000 g for 10 mins. Consequently, 50 μL of supernatant was mixed with 50 μL of NaOH (0.2N), and the absorbance was measure at 440 nm. Moreover, various protease inhibitors were applied to inhibit the protease activity of CraA, for instance, PMSF (serine protease), aprotinin (serine protease), pepstatin (aspartic acid protease), E-64 (cysteine protease) and EDTA (metalloprotease), so as to confirm the serine protease activity of CraA.

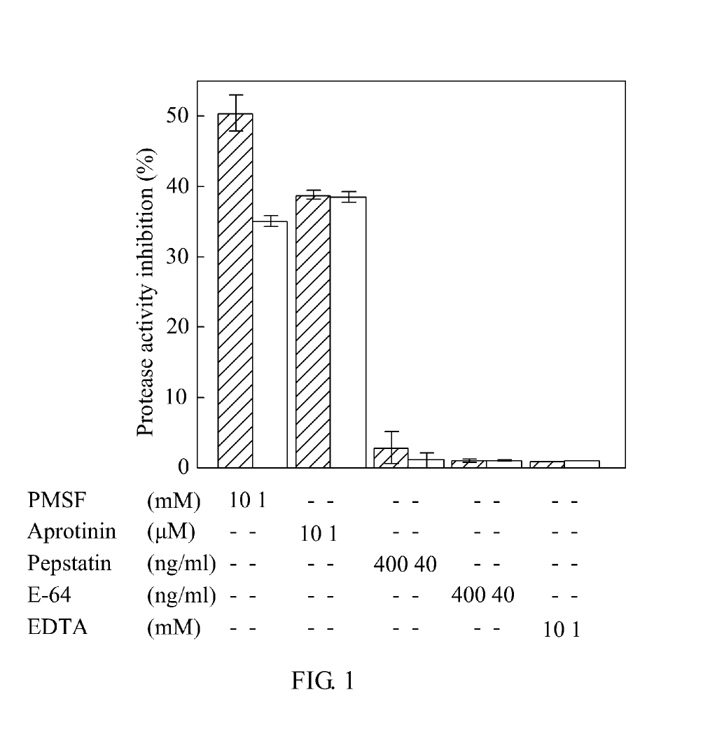

With reference to FIG. 1 for a schematic view of inhibition of protease activity of CraA in accordance with an embodiment of the present invention, the protease activity inhibition was shown after treatment of different kinds of proteases. Wherein, the protease activity of CraA is obviously inhibited by PMSF and aprotinin, which are serine protease inhibitors. However, the protease activity of CraA is not significantly inhibited by either pepstatin, E-64 or EDTA. That is, CraA indeed possesses serine protease activity, which may be inhibited by the serine protease inhibitors.

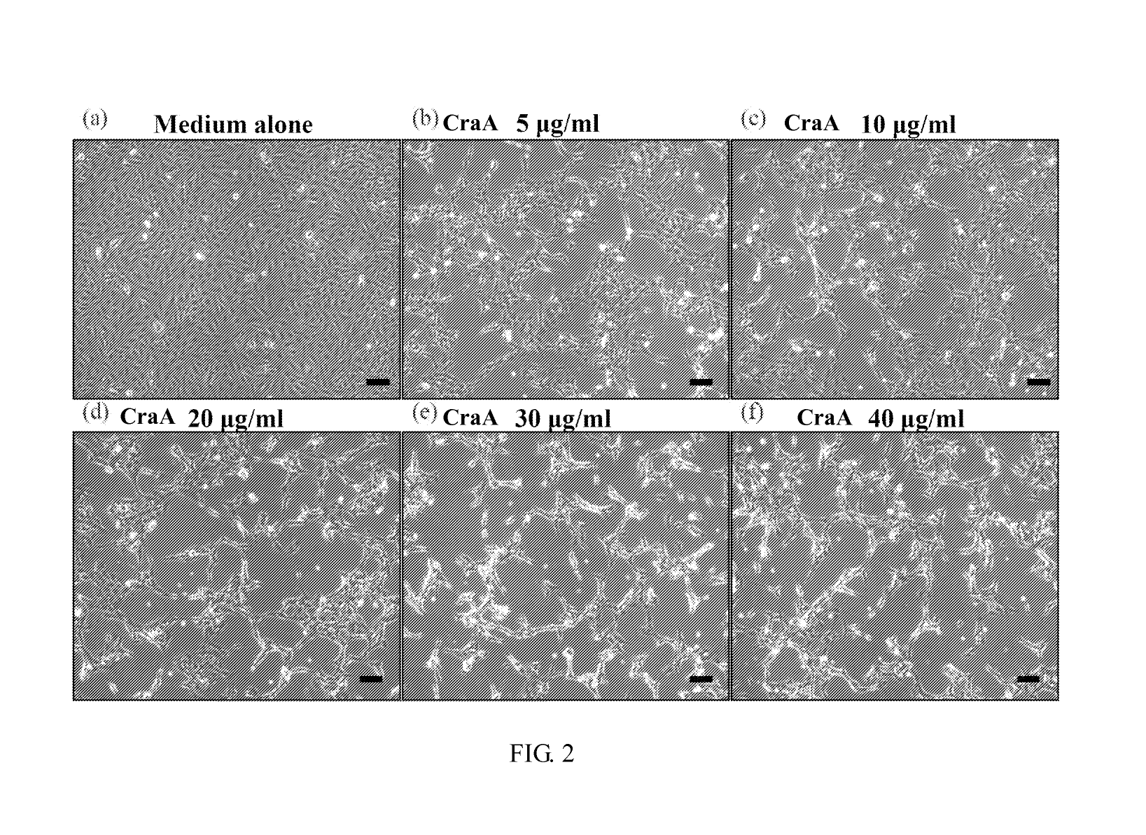

With reference to FIG. 2 is a schematic view of Beas2B cells treated with various doses of CraA in accordance with an embodiment of the present invention, 3×10⁵ Beas2B cells were seeded in a 6-well plate and treated with 0, 5, 10, 20, 30 and 40 μg/mL of CraA, respectively. Afterwards, the cells were observed by the bright field microscope. Referring to FIG. 2, Beas2B cells were increasingly dissociated from the bottom of the 6-well plate as the increasing concentration of the CraA. Accordingly, CraA induces the dissociation of the Beas2B cells.

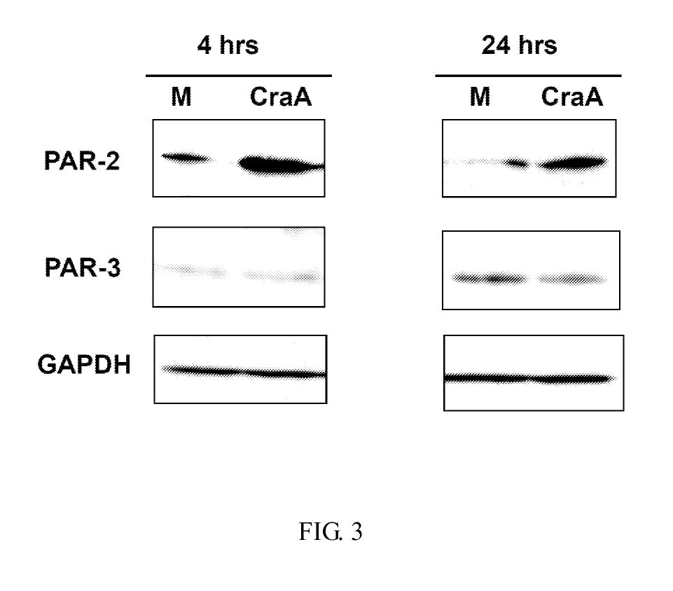

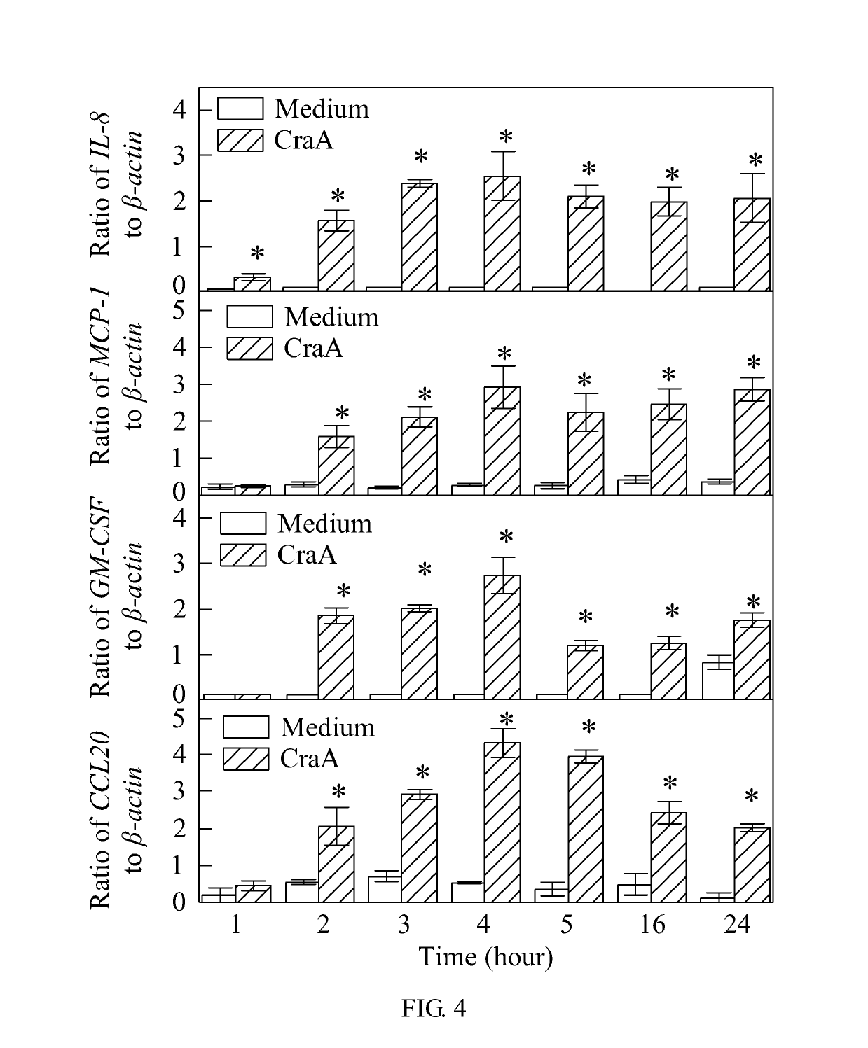

With reference to FIGS. 3-4 for a western blot of PAR-2 and PAR-3 protein in Beas2B cells treated with CraA and a quantification result of chemokines detected by RT-PCR in Beas2B cells treated with CraA in various time points, respectively, in accordance with an embodiment of the present invention, Beas2B cells were treated with CraA to confirm that activation of PAR receptor and downstream cytokines in the present cell model in response to the treatment of the serine protease type allergen, i.e. CraA.

Referring to FIG. 3, briefly, 3×10⁵ Beas2B cells were treated with 5 μg/mL of CraA for 4 or 24 hrs, respectively. Further, the cell lysate of the Beas2B cells treated with CraA was collected and analyzed by western blot analysis. As shown in the figure, the protein expression of PAR-2, rather than that of PAR-3, in Beas2B cells is induced by CraA comparing to the medium control (M), as in A549 cells. Namely, CraA activates PAR-2 after treating Beas2B cells with CraA for 4 or 24 hrs.

Referring to FIG. 4, the mRNA levels of various cytokines in response to treatment of CraA was measured by RT-PCR. To be brief, 3×10⁵ Beas2B cells were seeded in a 6-well plate and treated with 5 μg/mL of CraA for 1, 2, 3, 4, 5, 16 and 24 hrs, respectively. After the treatment, the cells were harvested by TRIzol (Invitrogen), and RNA of the cells was extracted. The extracted RNA was reverse-transcribed into cDNA by SuperScript polymerase (Invitrogen) according to the manufacturer's instruction. Moreover, IL-8, MCP-1, GM-CSF and CCL20 genes were amplified by RT-PCR and analyzed by 2% agarose gel. The expression levels of the above mentioned genes were normalized with the expression of β-actin. Wherein, IL-8, MCP-1, GM-CSF and CCL20 are chemotractors of neutrophil, monocyte, eosinophil and dendritic cell, respectively. As shown in the figure, the expression levels of the IL-8, MCP-1, GM-CSF and CCL20 are gradually increased as the increasing time of treatment of CraA. In addition, the highest ratios of IL-8, MCP-1, GM-CSF and CCL20 to β-actin are 2.5, 3, 3 and 4, respectively.

Gabexate Mesilate Protects Beas2B Cells from CraA

Gabexate mesilate is one of the serine protease inhibitors. Gabexate mesilate is extensively utilized in treating acute pancreatitis. A purpose of the present invention is to use gabexate mesilate as a nasal spray in preventing and treating allergy induced by serine protease type allergens. Therefore, the viability of the bronchus epithelial cells after treatment of gabexate mesilate is tested. In an embodiment, the solvent of gabexate mesilate may be distilled water, phosphate buffer solution (PBS) and the like. Preferably, gabexate mesilate is dissolved in distilled water.

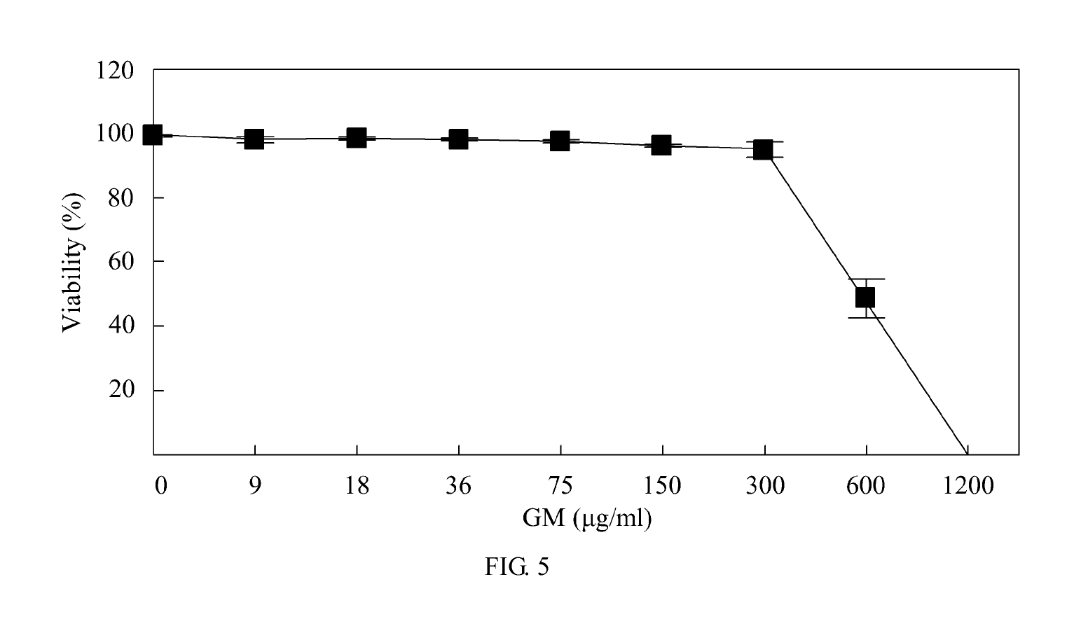

With reference to FIG. 5 for a schematic view of cell viability of Beas2B cells treated with gabexate mesilate (GM) in accordance with an embodiment of the present invention, 3×10⁵ Beas2B cells were seeded in a 3.5-cm dish with subsequently starvation for 16 hrs. After treating the cells with gabexate mesilate of various concentrations for 24 hrs, the cells were stained with trypan blue and counted the numbers of total cells, dead cells and survived cells so as to convert the numbers into a viability of the cells.

Referring to FIG. 5, the IC₅₀ of the cells to gabexate mesilate is 600 μg/mL, that is, the cell viability with respect to gabexate mesilate at concentration of 600 μg/mL is 50%. When the cells are treated with 1200 μg/mL of FOY, there are no survived cells. However, treating the cells with 300 μg/mL of gabexate mesilate would not affect the viability of the cells. Accordingly, the pharmaceutical effective amount of the composition comprising gabexate mesilate may be between 1-600 μg/mL, preferably between 10-300 μg/mL.

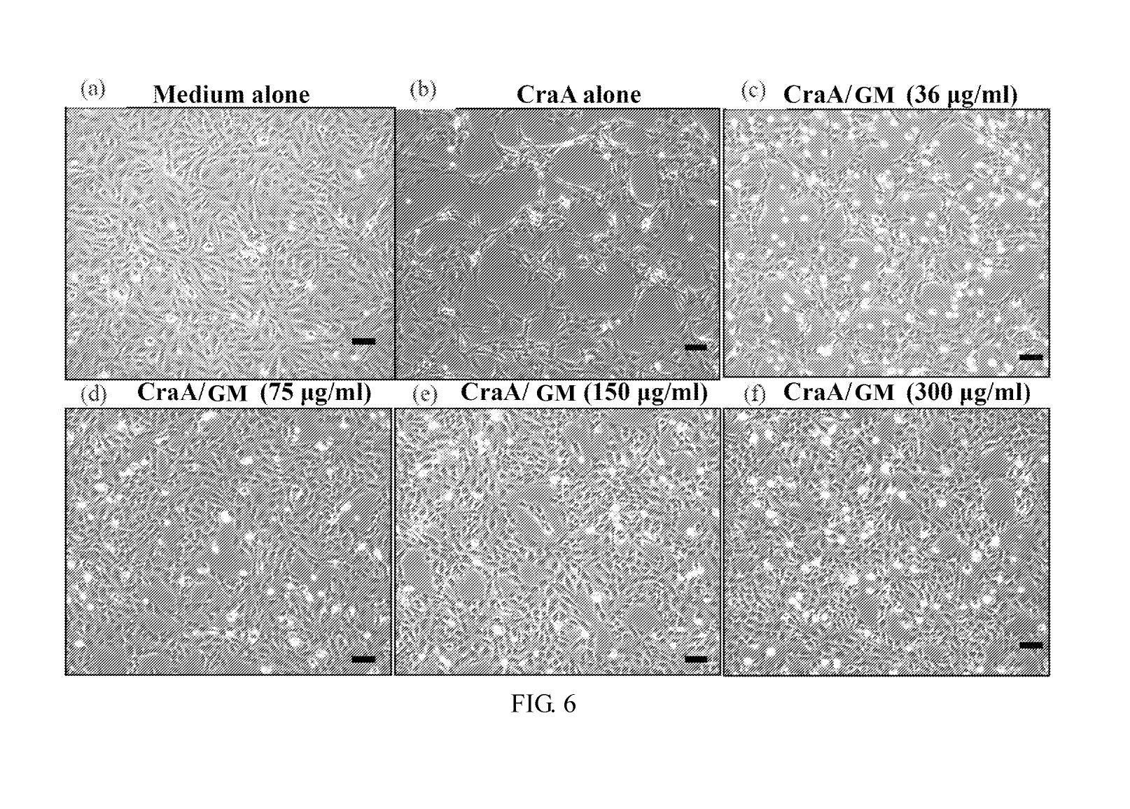

With reference to FIGS. 6-8 for a schematic view of protection effects of gabexate mesilate (GM) on Beas2B cells treated with CraA, a schematic view of chemokines detected by RT-PCR in Beas2B cells treated with CraA and gabexate mesilate and a quantification result of IL-8 and MCP-1 proteins in Beas2B cells treated with CraA and gabexate mesilate in accordance with an embodiment of the present invention, 3×10⁵ Beas2B cells were seeded in a 6-well plate and subsequently treated with gabexate mesilate for 30 mins. Afterwards, the cells were further treated with 5 μg/mL of CraA. Consequently, the cells were either observed by bright field microscope and analyzed the gene expression and the protein level of the cytokines, such as IL-8, MCP-1, GM-CSF and CCL20, as mentioned above.

Referring to FIG. 6, the cells treated with CraA only (b) are mostly dissociated from the bottom of the plate. However, treating the cells with either 36 (c), 75 (d), 150 (e) or 300 (f) μg/mL of gabexate mesilate reverse the dissociation of the cells. Moreover, as the treatment concentrations of gabexate mesilate increased, the attachment of the cells treated with CraA is gradually improved.

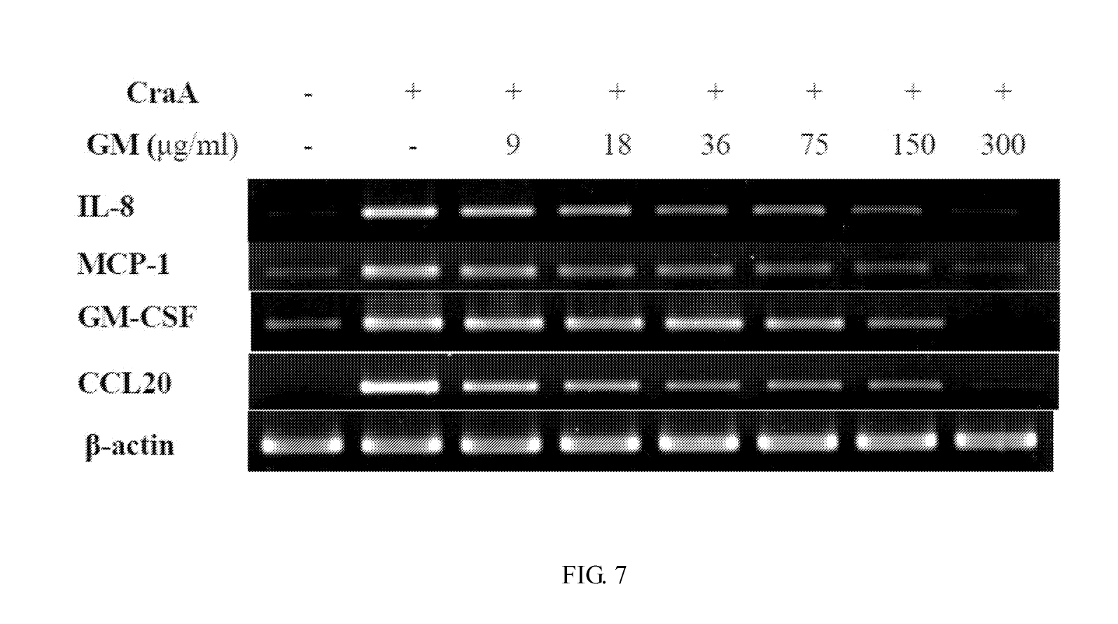

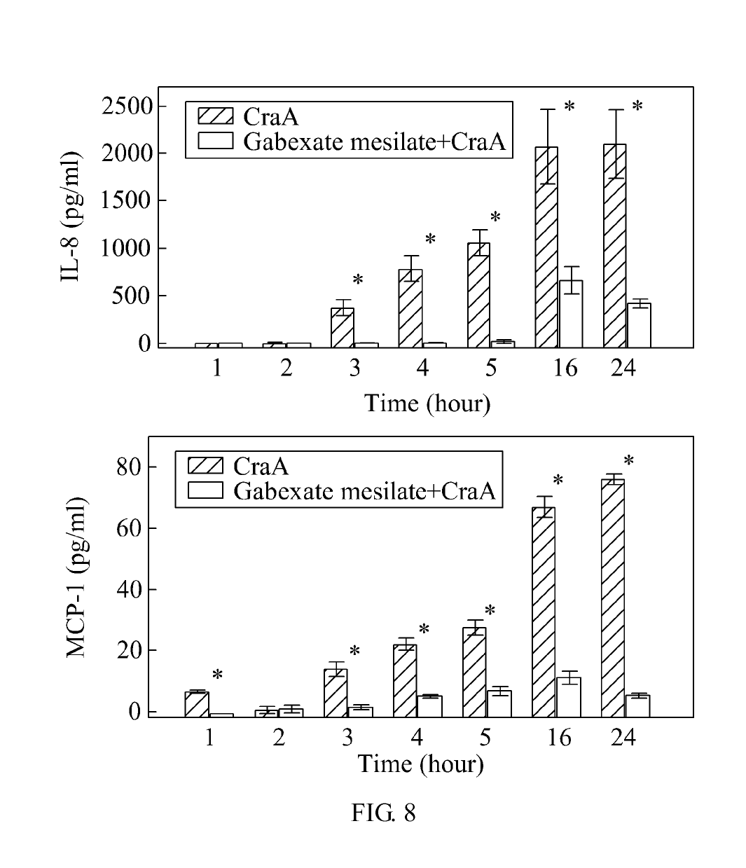

Referring to FIGS. 7-8, the expression levels of IL-8, MCP-1, GM-CSF and CCL20 genes are significantly increased in response to the treatment of CraA. However, gabexate mesilate obviously decreases the gene expression induced by CraA as the treatment doses of gabexate mesilate increased. Moreover, the protein levels of IL-8 and MCP-1 are significantly decreased in response to the treatment of gabexate mesilate during the time courses as well.

In summation of the description above, gabexate mesilate significantly reduces the dissociation of the bronchus epithelial cell, e.g. Beas2B cells used in the present invention, the activation of PAR and following immuno-inflammatory cascade. That is, topically applied the composition comprising gabexate mesilate to the airway may be a promising way to prevent or treat allergy induced by serine protease type allergens. Accordingly, administrating the composition comprising gabexate mesilate via nasal spray may be the easiest way with less side effects, comparing to conventional approaches, to prevent and treat allergy induced by a serine protease type allergen.

In vivo Treatment of Gabexate Mesilate CraA-Induced Asthma Murine Model

In an embodiment, for the asthma murine model, mice will be sensitized intraperitoneally on days 0, 7 and 14 with 40 μg of CraA containing 2 mg of alum and challenged intranasally on days 17, 19, 21, 23, 25 and 27. Twenty-four hours after the last challenge, airway responsiveness will be evaluated using whole-body plethysmography. For the treatment group, CraA-sensitized mice will receive intranasally with different doses of gabexate mesilate by nasal spray on days 17 through 27, once daily before challenge. Sham group will receive PBS only. PBS-sensitized and PBS-challenged mice will serve as negative control. Mice will be sacrificed on day 29 to evaluate all of allergic responses. Broncholaveolar larvage fluid, lung tissue, nares, trachea and spleen of each animal will be removed for examination after sacrifice. Accordingly, the mice with treatment of gabexate mesilate by nasal spray would alleviate the airway hyperresponsiveness caused by CraA. In addition, the elevation of immunoglobulin and cytokines would also be reduced treatment of gabexate mesilate by nasal spray.

In summation, the method of preventing and treating allergic airway diseases via intranasal gabexate mesilate shall prevent and treat allergy and inflammation induced by a serine protease type allergen. In addition, either the RNA level or protein level of the inflammatory cytokines were indeed reduced after gabexate mesilate treatment. Accordingly, treatment of gabexate mesilate by nasal spray protect the bronchus epithelial cells from the damages caused by the serine protease type allergen.

The present invention has been described with some preferred embodiments thereof and it is understood that many changes and modifications in the described embodiments can be carried out without departing from the scope and the spirit of the invention that is intended to be limited only by the appended claims.

What is claimed is:

1. A method of preventing and treating allergic airway diseases via intranasal gabexate mesilate, comprising:
administrating a pharmaceutically effective amount of a composition comprising a gabexate mesilate via nasal spray to prevent and treat allergy induced by a serine protease type allergen.

2. The method of claim 1, wherein the serine protease type allergen comprises a serine protease activity.

3. The method of claim 2, wherein the serine protease inhibitor prevents or inhibits the serine protease type allergen from activating of a protease-activated receptor (PAR).

4. The method of claim 3, wherein the PAR comprises PAR-2 and PAR-3.

5. The method of claim 2, wherein the serine protease type allergen induces a dissociation of epithelium cells from an extracellular matrix via the serine protease activity.

6. The method of claim 1, wherein a source of the serine protease type allergen comprises mite, mold, cat and cockroach.

7. The method of claim 6, wherein the serine protease type allergen being from mite comprises Der p 3, Der p 6 and Der p 9.

8. The method of claim 6, wherein the serine protease type allergen being from cockroach comprises American cockroach allergens (CraA).

9. The method of claim 1, wherein the composition further comprises distilled water, phosphate buffer solution (PBS) and the like.

10. The method of claim 1, wherein the pharmaceutically effective amount of the composition is between 1-600 μg/mL.

11. The method of claim 1, wherein the allergic airway diseases comprises sneezing, runny nose, clogged nose, postnasal drip, itching nose and asthma.
